SEQUENCE LISTING (32 sequences)

SEQ ID NO: 1 - protein, 353 aa, Dog
MDLQASLLPPGPNASNTSEGPDNLTSAGPPRRTGNVSYINIIMPSVFGTICLLGIIGNSTVIFAVVKKSKLHWCSNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGTVGCGIRLPNPDTDLYWFTLYQFFLAFALPFVVITAAYVRILQRMMSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 2 - protein, 353 aa, Ferret
MDLQASLLPTGPNASNTSDGPDNLTSAGPPPRRGSVSYINIIMPSVFGTICLLGIIGNSTVIFAVVKKSKLHWCSNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGTVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAISNAQTADEERTESKGT

SEQ ID NO: 3 - protein, 353 aa, Rhesus Monkey
MDLEASLLPTGPNTSNTSDGPDNLTSAGSPPRSGSVSYINIIMPSVFGTICLLGIIGNSMVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 4 - protein, 353 aa, Human
MDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 5 - protein, 353 aa, Rat
MDLQTSLLSTGPNASNISDGQDNLTLPGSPPRTGSVSYINIIMPSVFGTICLLGIVGNSTVIFAVVKKSKLHWCSNVPDIFIINLSVVDLLFLLGMPFMIHQLMGNGVWHFGETMCTLITAMDANSQFTSTYILTAMTIDRYLATVHPISSTKFRKPSMATLVICLLWALSFISITPVWLYARLIPFPGGAVGCGIRLPNPDTDLYWFTLYQFFLAFALPFVVITAAYVKILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRTVSNAQTADEERTESKGT

SEQ ID NO: 6 - protein, 353 aa, Mouse
MDLQASLLSTGPNASNISDGQDNFTLAGPPPRTRSVSYINIIMPSVFGTICLLGIVGNSTVIFAVVKKSKLHWCSNVPDIFIINLSVVDLLFLLGMPFMIHQLMGNGVWHFGETMCTLITAMDANSQFTSTYILTAMAIDRYLATVHPISSTKFRKPSMATLVICLLWALSFISITPVWLYARLIPFPGGAVGCGIRLPNPDTDLYWFTLYQFFLAFALPFVVITAAYVKILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRTVSNAQTADEERTESKGT

SEQ ID NO: 7 - DNA, 1062 bp, Dog
atggacctgcaagcctcgctgctgccccccggccccaacgccagcaacacctcggagggcccggacaacctcacctctgccgggccacctcgtcgcacagggaatgtctcctacatcaacatcatcatgccttccgtgttcggcaccatctgcctgctgggtatcatcgggaactccacagtcatcttcgcggtggtgaagaagtccaaactgcactggtgcagcaatgtccccgacatctttatcatcaacctctcggtggtagacctcctctttctcctgggcatgcccttcatgatccaccagctcatgggcaatggtgtttggcattttggagagaccatgtgcacactcatcacggccatggacgccaacagtcaattcaccagcacctacatcctgaccgccatggccattgaccgctacctggccactgtccaccccatctcctccaccaagttccggaagccctctgtggccaccctggtgatctgcctcctatgggccctctcattcatcagcatcacccccgtgtggctctacgctaggcttatccccttcccagggggcacagtgggctgtggcatccgcctgcccaacccagacactgacctttactggttcaccctgtaccagttcttcctggcctttgccctgcccttcgtggtcatcacagccgcgtatgtgaggatcctgcagcgcatgatgtcctcggtagcccctgcctctcaacgcagcatccggctgcggacaaagagggtgactcgcacggccattgccatctgcctggtcttcttcgtgtgctgggctccctactatgtgctacagttgacccagttgtccatcagccgcccgacactcacctttgtctacctgtacaacgcagccatcagcttgggctatgccaacagctgcctaaacccctttgtgtacatcgtgctctgtgagacattccgcaagcgcttggtcctgtcggtgaagcctgccgcccaggggcagcttcgagccgtcagcaatgctcagacagctgatgaggagaggacagaaagcaagggcacctga

SEQ ID NO: 8 - DNA, 1062 bp, Ferret
atggacctggaagcctcgctgctgcccactggccccaatgccagcaacacctcggatggccccgataacctcacctcggccgggccacctcctcgcagagggagtgtctcctacatcaacatcatcatgccttccgtgttcggcaccatctgcctcctgggcatcatcgggaactccacggtcatcttcgcggtggtgaagaagtccaaactgcactggtgcagcaacgtccccgacatcttcatcatcaacctctctgtggtggacctcctctttctcctgggcatgcccttcatgatccaccagctcatgggcaatggcgtctggcactttggagaaaccatgtgcacactgatcacggccatggatgccaacagtcagttcacgagcacctacatcctgacggccatggccattgaccgctacctggccactgtccaccccatctcttccaccaagttccggaagccctctgtggccaccctggtgatctgcctcctgtgggccctctccttcatcagcatcacccccgtgtggctctacgctaggctcatccccttcccggggggcacggtggggtgcggcatccgcctgcccaacccagacactgacctttactggttcaccctgtaccagttcttcctggcctttgccctgcccttcgtggtcatcactgccgcgtacgtgaggatcctgcagcgcatgacgtcctctgtagctcctgcctctcaacgcagcatccggctgcggaccaagagggtgactcgtacggccatcgccatctgcctggtcttctttgtgtgctgggctccctactatgtgctccagctgacccagctgtccatcagccgcccaaccctcacctttgtctacctgtacaacgccgccatcagcttgggctatgccaacagctgcctcaacccctttgtatacatcgtgctctgtgagacattccgcaaacgcttggtcctgtcggtgaagccagctgcccagggacagcttcgagccatcagcaacgcccagacagccgatgaggagaggacagaaagcaaaggcacctga

SEQ ID NO: 9 - DNA, 1062 bp, Rhesus monkey
atggacctggaagcctcgctgctgcccactggtcccaacaccagcaacacctctgatggccccgataacctcacctcggcaggatcacctcctcgctcagggagcgtctcctacatcaacatcatcatgccttcggtgttcggcaccatctgcctcctgggcatcatcgggaactccatggtcatcttcgcggtcgtgaagaagtccaagctgcactggtgcaacaatgtccccgacatcttcatcatcaacctctcggtggtggatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctacctggccaccgtccaccccatctcttccacaaagttccggaagccctctgtggccaccctggtgatctgcctcctgtgggccctctccttcatcagcatcacccccgtgtggttgtatgccagactcatccccttcccaggaggtgcagtgggctgcggcatccgcttgcccaacccggacactgacctttactggttcaccctgtaccagtttttcctggcctttgccctgcccttcgtggtcatcacggccgcatacgtgaggatcctgcagcgcatgacgtcctcagtggcccccgcctcccagcgcagcatccggctgcggacaaagagggtgacccgcacagccatcgccatctgcctggtcttctttgtgtgctgggcaccctactatgtgctacagctgacccagttgtccatcagccgcccgaccctcacctttgtctacctgtacaatgcggccatcagcttgggctacgccaacagctgcctcaacccctttgtgtacattgtgctctgcgagacgttccgcaaacgcttggtcctttcggtgaagcctgcagcccaggggcagcttcgcgctgtcagcaacgctcagacggctgacgaggagaggacagaaagcaaaggtacctga

SEQ ID NO: 10 - DNA, 1062 bp, Human
atggacctggaagcctcgctgctgcccactggtcccaacgccagcaacacctctgatggccccgataacctcacttcggcaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctacctggccactgtccaccccatctcttccacgaagttccggaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcatacgtgaggatcctgcagcgcatgacgtcctcagtggcccccgcctcccagcgcagcatccggctgcggacaaagagggtgacccgcacagccatcgccatctgtctggtcttctttgtgtgctgggcaccctactatgtgctacagctgacccagttgtccatcagccgcccgaccctcacctttgtctacttatacaatgcggccatcagcttgggctatgccaacagctgcctcaacccctttgtgtacatcgtgctctgtgagacgttccgcaaacgcttggtcctgtcggtgaagcctgcagcccaggggcagcttcgcgctgtcagcaacgctcagacggctgacgaggagaggacagaaagcaaaggcacctga

SEQ ID NO: 11 - DNA, 1062 bp, Rat
atggatctgcaaacctcgttgctgtccactggccccaatgccagcaacatctccgatggccaggataatctcacattgccggggtcacctcctcgcacagggagtgtctcctacatcaacatcattatgccttccgtgtttggtaccatctgtctcctgggcatcgtgggaaactccacggtcatctttgctgtggtgaagaagtccaagctacactggtgcagcaacgtccccgacatcttcatcatcaacctctctgtggtggatctgctcttcctgctgggcatgcctttcatgatccaccagctcatggggaacggcgtctggcactttggggaaaccatgtgcaccctcatcacagccatggacgccaacagtcagttcactagcacctacatcctgactgccatgaccattgaccgctacttggccaccgtccaccccatctcctccaccaagttccggaagccctccatggccaccctggtgatctgcctcctgtgggcgctctccttcatcagtatcacccctgtgtggctctacgccaggctcattcccttcccagggggtgctgtgggctgtggcatccgcctgccaaacccggacactgacctctactggttcactctgtaccagtttttcctggcctttgcccttccgtttgtggtcattaccgccgcatacgtgaaaatactacagcgcatgacgtcttcggtggccccagcctcccaacgcagcatccggcttcggacaaagagggtgacccgcacggccattgccatctgtctggtcttctttgtgtgctgggcaccctactatgtgctgcagctgacccagctgtccatcagccgcccgaccctcacgtttgtctacttgtacaacgcggccatcagcttgggctatgctaacagctgcctgaacccctttgtgtacatagtgctctgtgagacctttcgaaaacgcttggtgttgtcagtgaagcctgcagcccaggggcagctccgcacggtcagcaacgctcagacagctgatgaggagaggacagaaagcaaaggcacctga

SEQ ID NO: 12 - DNA, 1062 bp, Mouse
atggatctgcaagcctcgttgctgtccactggccccaatgccagcaacatctccgatggccaggataatttcacattggcggggccacctcctcgcacaaggagtgtctcctacatcaacatcatcatgccttcagtgtttggtaccatctgtctcctgggcattgtgggaaactccacagtcatttttgccgtggtgaagaaatccaagctgcactggtgcagcaacgtccctgacatcttcatcatcaacctctctgtggtggatctgcttttcctgctgggcatgcctttcatgatccaccagctcatgggtaatggtgtctggcactttggggaaaccatgtgcaccctcatcacagccatggacgccaacagtcagttcaccagcacctacatcctgactgctatggccattgaccgctacttggccaccgtccatcccatctcctccaccaagttccggaagccctccatggccaccctggtgatctgcctcctgtgggctctctcgttcattagcatcactcctgtgtggctctatgccaggcttatccccttcccagggggtgctgtgggctgtggcatccgcctaccaaacccagatactgatctttactggttcactctgtatcagtttttcctggccttcgcccttccgtttgtggtcatcactgctgcgtacgtgaaaatactacagcgcatgacgtcttcggtggccccagcctctcaacgcagcatccggcttcggacaaagagggtgacccgcacagccattgccatctgtctggtcttctttgtgtgctgggcgccctactacgtgctgcagctgacccagttgtccatcagccgcccgaccctcacattcgtctacctgtacaatgcggccatcagcttgggctatgccaacagctgcctcaatccctttgtgtacatagtactctgtgagacctttcgaaaacgcttggtgctgtcggtgaagcccgcggcccaggggcagcttcgcacggtcagcaatgctcagacagctgacgaggagaggacagaaagcaaaggcacctga

SEQ ID NO: 13 - protein, 18 aa, Artificial Sequence
  MCH-1R peptide
INIIMPSVFGTICLLGII

SEQ ID NO: 14 - DNA, 11 bp, Artificial Sequence
  MCH-1R nucleic acid region
aacctcacctc

SEQ ID NO: 15 - DNA, 21 bp, Artificial Sequence
  MCH-1R nucleic acid region
ttcatcagcatcacccccgtg

SEQ ID NO: 16 - DNA, 20 bp, Artificial Sequence
  MCH-1R nucleic acid region
caacccagacactgaccttt

SEQ ID NO: 17 - DNA, 18 bp, Artificial Sequence
  MCH-1R nucleic acid region
ctgcccttcgtggtcatc

SEQ ID NO: 18 - DNA, 17 bp, Artificial Sequence
  MCH-1R nucleic acid region
ctcacctttgtctacct

SEQ ID NO: 19 - DNA, 23 bp, Artificial Sequence
  Oligonucleotide primer
ggnatgccnttyhtnathcayca

SEQ ID NO: 20 - DNA, 23 bp, Artificial Sequence
  Oligonucleotide primer
arytgnadnacrtrrtanggngc

SEQ ID NO: 21 - DNA, 25 bp, Artificial Sequence
  Oligonucleotide primer
cacggccatggatgccaatagtcag

SEQ ID NO: 22 - DNA, 25 bp, Artificial Sequence
  Oligonucleotide primer
aagaagaccaggcagatggcgatgg

SEQ ID NO: 23 - DNA, 25 bp, Artificial Sequence
  Oligonucleotide primer
tcaccagcacctacatcctgaccgc

SEQ ID NO: 24 - DNA, 25 bp, Artificial Sequence
  Oligonucleotide primer
cagggcaaaggccaggaaaaactgg

SEQ ID NO: 25 - DNA, 25 bp, Artificial Sequence
  Oligonucleotide primer
agtcaacacttacgccaagagctga

SEQ ID NO: 26 - DNA, 27 bp, Artificial Sequence
  Oligonucleotide primer
cgcctccatcaacaaactttcttgtat

SEQ ID NO: 27 - DNA, 28 bp, Artificial Sequence
  Oligonucleotide primer
ttagttttaccgttttcgagctgctcta

SEQ ID NO: 28 - DNA, 25 bp, Artificial Sequence
  Oligonucleotide primer
gattgggggtaaataacagaggtgg

SEQ ID NO: 29 - DNA, 23 bp, Artificial Sequence
  Oligonucleotide primer
ttcatgatccaccagctcatggg

SEQ ID NO: 30 - DNA, 23 bp, Artificial Sequence
  Oligonucleotide primer
tcaggtgcctttgctttctgtcc

SEQ ID NO: 31 - DNA, 36 bp, Artificial Sequence
  Oligonucleotide primer
cggaattcgccgccatggacctgggagcctcgctgc

SEQ ID NO: 32 - DNA, 33 bp, Artificial Sequence
  Oligonucleotide primer
gctctagatcaggtgcctttgctttctgtcctc

(19) United States
(12) Patent Application Publication
(10) Pub. No.: US 2004/0248129 A1
(43) Pub. Date: Dec. 9, 2004

(54) Rhesus monkey, dog and feeret melanin-concentrating hormone type 1 receptor

(76) Inventors: Carina Tan, Metuchen, NJ (US); Andreas W. Sailer, Edison, NJ (US); Jie Pan, Plainsboro, NJ (US); Hideki Sano, Nagareyama (JP)

Correspondence Address: MERCK AND CO INC, P O BOX 2000, RAHWAY, NJ, 070650907

(21) Appl. No.: 10/488,724

(22) PCT Filed: Sep. 20, 2002
(86) PCT No.: PCT/US02/29932

Related U.S. Application Data
(60) Provisional application No. 60/324,419, filed on Sep. 24, 2001.

(51) Int. Cl. C12Q 1/68; C07H 21/04; C07K 14/72

(52) U.S. Cl. 435/6; 435/69.1; 435/320.1; 435/325; 530/350; 536/23.5

(57) ABSTRACT

The present invention features polypeptide and nucleic acid sequences related to the dog, ferret and rhesus monkey MCH-1R. Preferred polypeptides and nucleic acid contain a region common to the dog, ferret and rhesus monkey MCH-1R, which is not present in the human, rat, or mouse MCH-1R.

### CROSS-REFERENCE TO RELATED APPLICATIONS

The present application claims priority to provisional application U.S. Serial No. 60/324,419, filed Sep. 24, 2001, hereby incorporated by reference herein.

### BACKGROUND OF THE INVENTION

The references cited in the present application are not admitted to be prior art to the claimed invention.

Neuropeptides present in the hypothalamus play a major role in mediating the control of body weight. (Flier, et al., 1998. Cell, 92, 437-440.) Melanin-concentrating hormone (MCH) is a cyclic 19-amino acid neuropeptide synthesized as part of a larger pre-prohormone precursor in the hypothalamus which also encodes neuropeptides NEI and NGE. (Nahon, et al., 1990. Mol. Endocrinol. 4, 632-637.) MCH was first identified in salmon pituitary, and in fish MCH affects melanin aggregation thus affecting skin pigmentation. In trout and in eels MCH has also been shown to be involved in stress induced or CRF-stimulated ACTH release. (Kawauchi, et al., 1983. Nature 305, 321-323.)

In humans two genes encoding MCH have been identified that are expressed in the brain. (Breton, et al., 1993. Mol. Brain Res. 18, 297-310.) In mammals MCH has been localized primarily to neuronal cell bodies of the hypothalamus which are implicated in the control of food intake, including perikarya of the lateral hypothalamus and zona inertia. (Knigge, et al., 1996. Peptides 17, 1063-1073.)

Pharmacological and genetic evidence suggest that the primary mode of MCH action is to promote feeding (orexigenic). MCH mRNA is up regulated in fasted mice and rats and in the ob/ob mouse. (Qu, et al., 1996. Nature 380, 243-247.) Injection of MCH centrally (ICV) stimulates food intake and MCH antagonizes the hypophagic effects seen with a melanocyte stimulating hormone (α(MSH). (Qu, et al., 1996. Nature 380, 243-247.) MCH deficient mice are lean, hypophagic and have increased metabolic rate. (Shimada, et al., 1998. Nature 396, 670-673.) Transgenic present in a sample or preparation. In preferred embodiments, the purified polypeptide represents at least about 50%, at least about 75%, or at least about 95% of the total protein in a sample or preparation. Reference to “purified polypeptide” does not require that the polypeptide has undergone any purification and may include, for example, chemically synthesized polypeptide that has not been purified.

Another aspect of the present invention describes a purified nucleic acid comprising a nucleotide sequence region encoding for SEQ. ID. NO. 13. A “purified nucleic acid” represents at least 10% of the total nucleic acid present in a sample or preparation. In preferred embodiments, the purified nucleic acid represents at least about 50%, at least about 75%, or at least about 95% of the total nucleic acid in a sample or preparation. Reference to “purified nucleic acid” does not require that the nucleic acid has undergone any purification and may include, for example, chemically synthesized nucleic acid that has not been purified.

Another aspect of the present invention describes a purified nucleic acid having a nucleotide sequence region at least eleven nucleotides in length that is common to the dog, ferret, and rhesus monkey MCH-1R, but is not present in.the. : human, mouse or rat MCH-IR. Examples of such sequences are provide by SEQ. ID. NO. 14, SEQ. ID. NO. 15, SEQ. ID. NO. 16, SEQ. ID. NO. 17, SEQ. ID. NO. 18 and the complements thereof.

Another aspect of the present invention describes a recombinant nucleic acid comprising a nucleotide sequence that either (a) encodes a polypeptide comprising the amino acid sequence of SEQ. ID. NO. 13; or (b) provides a sequence region that is common to the dog, ferret, and rhesus monkey MCH-1R, but is not present in the human, rat, or mouse MCH-1R.

A “recombinant nucleic acid” is a nucleic acid containing one or more regions not naturally associated with each other. Recombinant nucleic acid can be present in a genome or outside of the genome.

Another aspect of the present invention describes a recombinant cell comprising an expression vector that comprises a nucleotide sequence encoding an amino acid region of SEQ. ID. NO. 13. The sequence is coupled to a promoter recognized by the cell.

Another aspect of the present invention describes a recombinant cell made by a process involving use of an expression vector comprising a nucleotide sequence encoding an amino acid region of SEQ. ID. 13.

Another aspect of the present invention describes a method of measuring the ability of a compound to affect MCH-1R activity. The method comprises the steps of: (a) contacting a recombinant cell with the compound, wherein the cell comprises a recombinant nucleic acid expressing a functional MCH-1R; and (b) measuring MCH-1R activity. The functional MCH-1R expressed by the cell has an amino acid sequence region of SEQ. ID. NO. 13.

Another aspect of the present invention describes a method of preparing a MCH-1R polypeptide. The method comprises the step of growing a recombinant cell under conditions where the polypeptide is expressed from recombinant nucleic acid. The recombinant nucleic acid encodes a functional MCH-1R containing the amino acid sequence region of SEQ. ID. NO. 13.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIGS. 1A and 1B provide an amino acid sequence comparison of the dog (SEQ. ID. NO. 1), ferret (SEQ. ID. NO. 2), rhesus monkey (SEQ. ID. NO. 3), human (SEQ. ID. NO. 4), rat (SEQ. ID. NO. 5), and mouse (SEQ. ID. NO. 6) MCH-1R.

FIGS. 2A, 2B, and 2C provide a comparison of cDNA sequences for the dog (SEQ. ID. NO. 7), ferret (SEQ. ID. NO. 8), rhesus monkey (SEQ. ID. NO. 9), human (SEQ. ID. NO. 10), rat (SEQ. ID. NO. 11), and mouse (SEQ. ID. NO. 12) MCH-1R.

FIG. 3 illustrates the ability of MCH and [Phe¹³,Tyr¹⁹]-MCH to cause a dose-dependent increase in intracellular calcium levels in HEK293T cells transiently expressing rhesus MCH-1R.

FIG. 4 illustrates the ability of MCH and [Phe¹³,Tyr¹⁹]-MCH to dose-dependently inhibit [¹²⁵I]- MCH binding to the rhesus MCH-1R expressed on HEK293T cells.

### DETAILED DESCRIPTION OF THE INVENTION

The identification of the MCH1R in the dog, ferret, and rhesus monkey provides MCH-1R amino acid and nucleic acid sequence information and a model for evaluating the effect of compounds that modulate MCH1R activity. Dog, ferret and rhesus monkey MCH-1R sequence information have a variety of uses including being used to produce MCH-1R polypeptides and nucleic acids.

A preferred use of MCH-1R amino acid and nucleic acid sequences is to produce MCH-1R functional polypeptides that can be used in the initial identification of compounds binding to MCH-1R and modulating MCH-1R activity. The in vivo activity of such compounds can then be evaluated using, for example, a dog, ferret or rhesus monkey.

Compounds modulating MCH-1R activity have a variety of different uses including utility as a tool to further study MCH1R activity and as an agent to achieve a beneficial effect in a patient. Modulating MCH-1R activity includes evoking a response at the receptor and altering a response evoked by a MCH-1R agonist or antagonist.

Beneficial effects of modulating MCH-1R activity include achieving one or more of the following in a patient: weight loss, weight gain, cancer treatment (e.g., colon or breast), pain reduction, diabetes treatment, stress reduction and sexual dysfunction treatment. A patient is a mammal, preferably a human. Reference to patient does not necessarily indicate the presence of a disease or disorder. The term patient includes subjects treated prophylactically and subjects afflicted with a disease or disorder.

Preferably, MCH-1R activity is modulated to treat diabetes, to obtain a weight loss, or to obtain a weight gain. Diabetes mellitus can be treated by, for example, one or both of the following: enhancing glucose tolerance and decreasing insulin resistance.

Excessive weight is a contributing factor to different diseases including hypertension, diabetes, dyslipidemias, cardiovascular disease, gall stones, osteoarthritis and certain forms of cancers. Bringing about a weight loss can be used, for example, to reduce the likelihood of such diseases and as part of a treatment for such diseases. Weight reduction can be achieved by, for example, one or more of the following: reducing appetite, increasing metabolic rate, reducing fat intake and reducing carbohydrate craving.

Increasing weight is particularly useful for a patient having a disease or disorder, or under going a treatment, accompanied by weight loss. Examples of diseases or disorders accompanied by weight loss include anorexia, AIDS, wasting, cachexia, and frail elderly. Examples of treatments accompanied by weight loss include chemotherapy and radiation therapy.

### MCH-1R Polypeptides

MCH-1R polypeptides featured herein contain a region of SEQ. ID. NOs. 1, 2, or 3 that is at least 9 contiguous amino acids in length. The MCH-1R polypeptide can be made up of only MCH-1R sequences from SEQ. ID. NOs. 1-3 or can be a chimeric polypeptide. MCH-1R polypeptides have a variety of uses, such as providing a component for a functional receptor; being used as an immunogen to produce antibodies binding to MCH-1R; being used as a target to identify compounds binding to the MCH-1R; and being used in assays to measure the ability of a compound to affect MCH-1R activity.

Chimeric polypeptides contain one or more regions from MCH-1R and one or more regions from MCH-1R. The region(s) not from MCH-1R can be used, for example, to achieve a particular purpose or to produce a polypeptide that can substitute for MCH-1R or a fragment thereof. Particular purposes that can be achieved using MCH-1R polypeptides that are chimeric include providing a marker for isolation, functional analysis of different receptor regions, enhancing an immune response, and altering G-protein coupling.

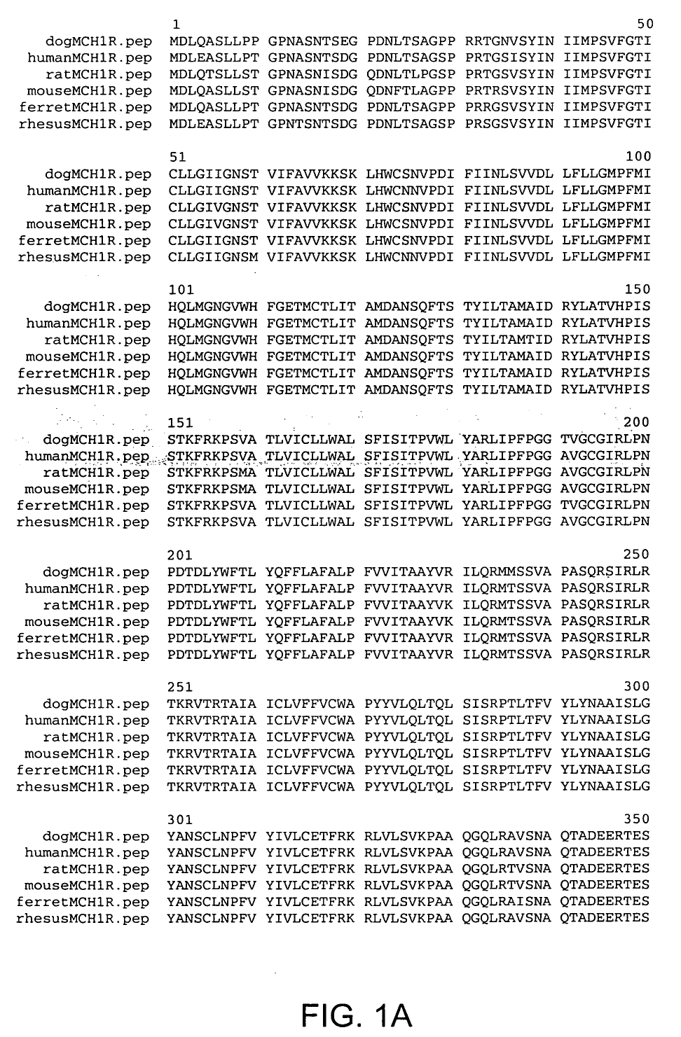

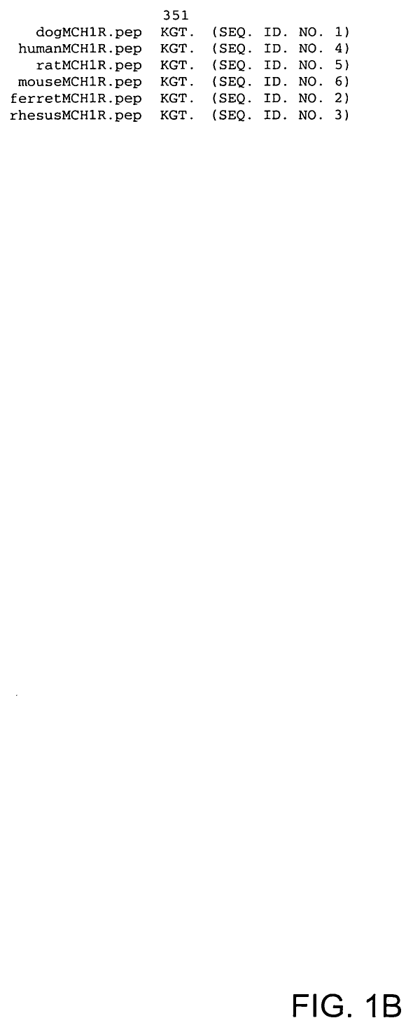

MCH-1R polypeptides may contain a sequence region that is unique to either the dog, ferret, or rhesus monkey, or is present in two or more of these different animals. FIGS. 1A and 1B illustrate an amino acid sequence comparison of the dog, ferret, rhesus monkey, human, rat and mouse MCH-1R. The sequence comparison provided in FIG. 1 identifies unique and common amino acid sequences.

A “unique” amino acid sequence differs from at least the human, rat, and mouse sequence, and preferably also from either the dog and ferret, dog and rhesus monkey, ferret and rhesus monkey, or dog and rhesus monkey. In an embodiment of the present invention, the MCH-1R polypeptide comprises or consists of an amino acid sequence at least 9 bases in length that is unique for at least one of SEQ. ID. NOs. 1-3. In additional embodiments, the unique sequence is at least 18 amino acids in length, at least 27 amino acids in length, or at least 54 amino acids in length.

A “common” amino acid sequence is with respect to at least two of the dog, ferret, and rhesus monkey sequences. In an embodiment of the present invention, the MCH-1R polypeptide has an amino acid sequence region at least 18 amino acids in length that is common to the dog, ferret, and rhesus monkey MCH-1R, but is not present in the human MCH-1R. An example of such a polypeptide is provided by SEQ. ID. NO. 13: INIIMPSVFGTICLLGII.

Additional MCH-1R polypeptides described herein include functional G-protein receptors that respond to MCH and (1) have a sequence similarity of at least about 98% with either SEQ. ID. NOs. 1, 2 or 3; or (2) provide a sequence with up to about 10 alterations from SEQ. ID. NOs. 1, 2, or 3. Sequence similarity for polypeptides can be determined by BLAST. (Altschul, et al., 1997. Nucleic Acids Res. 25, 3389-3402, hereby incorporated by reference herein.) In one embodiment sequence similarity is determined using tBLASTn search program with the following parameters: MATRIX:BLOSUM62, PER RESIDUE GAP COST: 11, and Lambda ratio: 1.

Alterations to amino acid sequences are additions, deletions, and substitutions. In different embodiments the MCH-1R polypeptide has 1, 2, 3, 4, 5, 6, 7, 8, 9, or 10 alterations from SEQ. ID. NOs. 1, 2, or 3.

Polypeptides can be produced using standard techniques including those involving chemical synthesis and those involving biochemical synthesis. Techniques for chemical synthesis of polypeptides are well known in the art. (See e.g., Vincent, in Peptide and Protein Drug Delivery, New York, N.Y., Dekker, 1990.)

Biochemical synthesis techniques for polypeptides are also well known in the art. Such techniques employ a nucleic acid template for polypeptide synthesis. The genetic code providing the sequences of nucleic acid triplets coding for particular amino acids is well known in the art. (See, e.g., Lewin GENES IV, p. 119, Oxford University Press, 1990.) Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2nd Edition, Cold Spring Harbor Laboratory Press, 1989.

Functional MCH-1R

Functional MCH-1R transduces a G-protein coupled intracellular signal upon ligand binding. The identification of the amino acid and nucleic acid MCH-1R sequences provide tools for producing dog, ferret, and rhesus monkey MCH-1R, for obtaining MCH-1R from other sources, for producing MCH-1R chimeric G-protein coupled receptors, and for producing functional derivatives of the dog, ferret and rhesus monkey MCH-1R.

MCH-1R polypeptides from different sources can be identified and obtained based on their sequence similarity to the dog, ferret, or rhesus monkey MCH-1R. The amino acid and nucleic acid sequences of the dog, ferret, and rhesus monkey MCH-1R can be used to help identify and obtain additional MCH-1R polypeptides. For example, SEQ. ID. NOs. 1, 2 or 3 can be used to design degenerative nucleic acid probes or primers for identifying and cloning nucleic acid encoding for a MCH-1R polypeptide, and SEQ. ID. NOs. 7, 8, or 9, the complement of SEQ. ID. NOs. 7, 8, or 9, and fragments thereof, can be used under conditions of moderate stringency to identify and clone nucleic acid encoding MCH-1R polypeptides.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2 d Edition, Cold Spring Harbor Laboratory Press, 1989.

Starting with MCH-1R obtained from a particular source, derivatives can be produced having receptor activity. Such derivatives include polypeptides with amino acid substitutions, additions and deletions. Changes to MCH1R to produce a derivative having essentially the same properties should be made outside of the MCH-1R binding domain and in a manner not altering the tertiary structure. The ability of a polypeptide to have MCH-1R activity can be confirmed using techniques such as those measuring G-protein activity.

Differences in naturally occurring amino acids are due to different R groups. An R group affects different properties of an amino acid such as physical size, charge, and hydrophobicity. Amino acids can be divided into different groups as follows: neutral and hydrophobic (alanine, valine, leucine, isoleucine, proline, tyrptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolor amino acids in the interior of a polypeptide then glutamate because of its long aliphatic side chain. (See, Ausubel, Current Protocols in Molecular Biology, John Wiley, 1987-1998, Supplement 33 Appendix IC.)

MCH-1R Antibodies

Antibodies recognizing MCH-1R can be produced using a polypeptide containing SEQ. ID. NOs. 1, 2, or 3, or a fragment thereof as an immunogen. In an embodiment of the present invention, a polypeptide used as an immunogen consists of a polypeptide of SEQ. ID. NOs. 1, 2, or 3 or a fragment at least 9 amino acids in length.

Antibodies to MCH-1R have different uses such as being used to identify the presence of MCH-1R and to isolate MCH-1R polypeptides. Identifying the presence of MCH-1R can be used, for example, to identify cells producing MCH-1R. Such identification provides an additional source of MCH-1R and can be used to distinguish cells known to produce MCH-1R from cells that do not produce MCH-1R.

Techniques for producing and using antibodies are well known in the art. Examples of such techniques are described in Ausubel, Current Protocols in Molecular Biology, John Wiley, 1987-1998, Harlow, et al., Antibodies, A Laboratory Manual, Cold Spring Harbor Laboratory, 1988, and Kohler, et al., Nature 256:495-497, 1975.

Binding Assay

MCH-1R or a fragment thereof can be used in binding studies to identify compounds binding to the receptor. Such studies can be performed using different formats including competitive and non-competitive formats.

The particular MCH-1R sequence involved in ligand binding can be identified using labeled compounds that bind to the receptor and different receptor fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided or mutated to further locate the binding region. Fragments used for binding studies can be generated by recombinant nucleic acid techniques.

Preferably, binding studies are performed using MCH1R expressed from a recombinant nucleic acid. In an embodiment of the present invention, a recombinantly expressed MCH-1R consists of the amino acid sequence of SEQ. ID. NO. 1, SEQ. ID. NO.2, or SEQ. ID. NO.3.

Binding assays can be performed using individual compounds or preparations containing different numbers of compounds. A preparation containing different numbers of compounds having the ability to bind to MCH-1R can be divided into smaller groups of compounds that can be tested to identify the compound(s) binding to MCH-1R.

Binding assays can be performed using MCH-1R present in different environments. Such environments include, for example, cell extracts and purified cell extracts containing MCH-1R; and also include, for example, the use of a purified MCH-1R polypeptide which is introduced into a different environment.

The identification of MCH as an agonist for MCH-1R provides a means for producing receptor activity, and provides a target for binding to the receptor and for stimulating receptor activity. MCH agonists can be designed based on the structure of MCH. Examples of MCH agonists include human MCH, salmon MCH, and derivatives thereof. Suitable derivatives can be identified empirically, for example, by deleting or substituting one or more amino acids of human MCH and testing the resulting polypeptide. Techniques for producing a polypeptide of a particular sequence are well known in the art. (Phe¹³Tyr¹⁹)-MCH is an example of an MCH derivative that binds MCH-1R.

Different types of labels for MCH agonists can be employed. Examples of such labels include radiolabels, luminescent molecules, haptens and enzyme substrates. The ability of a particular label to interfere with binding can be determined by comparing the ability of MCH containing the particular label to compete against unlabeled MCH.

Functional Assays

Assays involving a functional G-protein receptor containing one or more MCH-1R regions can be employed for different purposes such as selecting for compounds active at MCH-1R, evaluating the ability of a compound to affect receptor activity, and mapping the activity of different MCH-1R regions. MCH-1R activity can be measured using different techniques such as detecting a change in the intracellular conformation of MCH-1R, measuring G-protein activity, or measuring the level of intracellular messengers.

Recombinantly expressed receptor can be used to facilitate determining whether a compound is active at that receptor or another receptor. For example, MCH-1R can be expressed by an expression vector in a cell line such as HEK 293, COS 7, or CHO, not normally expressing the receptor, wherein the same cell line without the expression vector or with an expression vector not encoding MCH-1R can act as a control.

Techniques for measuring different G-protein activities, such as Gi, Gs, and Gq are well known in the art. Gi and Gs activity can be measured using techniques such as a melonaphore assay, assays measuring cAMP production, assays measuring inhibition of cAMP accumulation, and assays measuring binding of ³⁵s-GTP. cAMP can be measured using different techniques such as a radioimmunoassay and indirectly by cAMP responsive gene reporter proteins.

Gq activity can be measured using techniques such as those measuring intracellular Ca²⁺. Examples of techniques well known in the art that can be employed to measure Ca²⁺ include the use of dyes such as Fura-2 and the use of Ca²⁺-bioluminescent sensitive reporter proteins such as aequorin. An example of a cell line employing aequorin to measure G-protein activity is HEK293/aeq17. (Button, et al., 1993. Cell Calcium 14, 663-671, and Feighner, et al., 1999. Science 284, 2184-2188, both of which are hereby incorporated by reference herein.)

Chimeric MCH-1R can be used to assay for compounds active at the receptor and to obtain information concerning different regions of the receptor. A chimeric MCH-1R receptor contains an N-terminal extracellular domain; a transmembrane domain made up of transmembrane regions (preferably 7 transmembrane regions), extracellular loop regions, and intracellular loop regions; and an intracellular carboxy terminus domain; where one or more domains comprises a unique or common region of SEQ. ID. NOs. 1-3 of at least 18 contiguous amino acids. In different embodiments, a chimeric MCH-1R contains the extracellular domain of MCH-1R present in either SEQ. ID. NOs. 1, 2, or 3; the unique or common region contains at least 36 contiguous amino acids present in SEQ. ID. NOs. 1, 2 or 3; or a common region having the sequence of SEQ. ID. NO. 13 is present.

The specificity of G-protein coupling is determined by intracellular domain(s). Chimeric MCH-1R can be produced to functionally couple to a desired G-protein. Techniques for producing chimeric receptors and measuring G-protein coupled responses are provided in, for example, International Publication Number WO 97/05252, U.S. Pat. No. 5,981,195, and U.S. Pat. No. 5,264,565.

Functional assays can be performed using individual compounds or preparations containing different compounds. A preparation containing different compounds where one or more compounds affect MCH1R activity can be divided into smaller groups of compounds to identify the compound(s) affecting MCH-1R activity.

Functional assays can be performed using recombinantly produced MCH-1R present in different environments. Such environments include, for example, cells expressing recombinant nucleic acid encoding a functional MCH-1R, cell extracts and purified cell extracts containing the MCH-1R expressed from recombinant nucleic acid and an appropriate membrane for the polypeptide; and the use of a purified MCH-1R produced by recombinant means that is introduced into a different environment suitable for measuring G-protein activity.

Screening for MCH-1R receptor active compounds is facilitated through the use of a MCH agonist in the assay. The use of a MCH agonist in a screening assay provides for MCH-1R activity. The effect of test compounds on such activity can be measured to identify, for example, allosteric modulators and antagonists.

### MCH-1R Nucleic Acid

MCH-1R nucleic acid featured herein includes nucleic acid containing a region encoding a MCH-1R polypeptide having a unique or common region at least 18 amino acids in length that is present in SEQ. ID. NOs. 1, 2, or 3, and nucleic acid containing a unique or common region of at least 11 contiguous bases that is present in at least one of SEQ. ID. NOs. 7, 8, or 9, or the complement thereof. MCH-1R nucleic acid have a variety of uses, such as being used as a hybridization probe or PCR primer to identify the presence of MCH-1R nucleic acid; being used as a hybridization probe or PCR primer to identify nucleic acid encoding receptors related to MCH-1R and being used for recombinant expression of MCH-1R polypeptides.

MCH-1R nucleic acid may be associated with nucleic acid not from MCH-1R. Regions in MCH-1R nucleic acid that do not encode a MCH-1R segment or are not found in SEQ. ID. NOs. 7, 8, 9, or the complement thereof, if present, are preferably chosen to achieve a particular purposes. Examples of additional regions that can be used to achieve a particular purpose include capture regions that can be used as part of a sandwich assay, reporter regions that can be probed to indicate the presence of the nucleic acid, expression vector regions, and regions encoding other polypeptides.

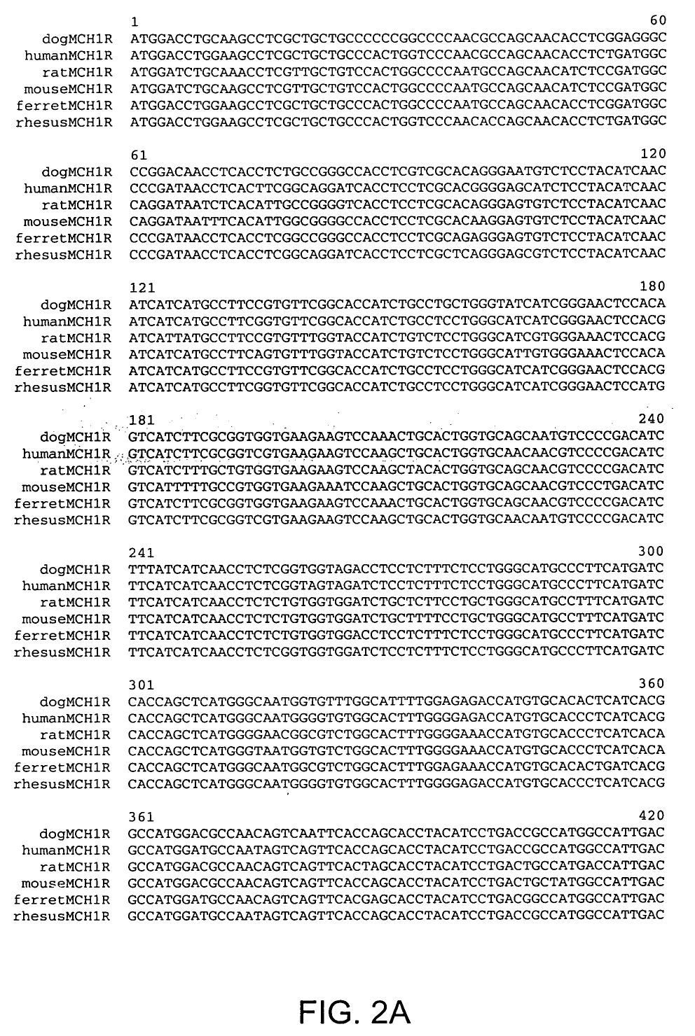

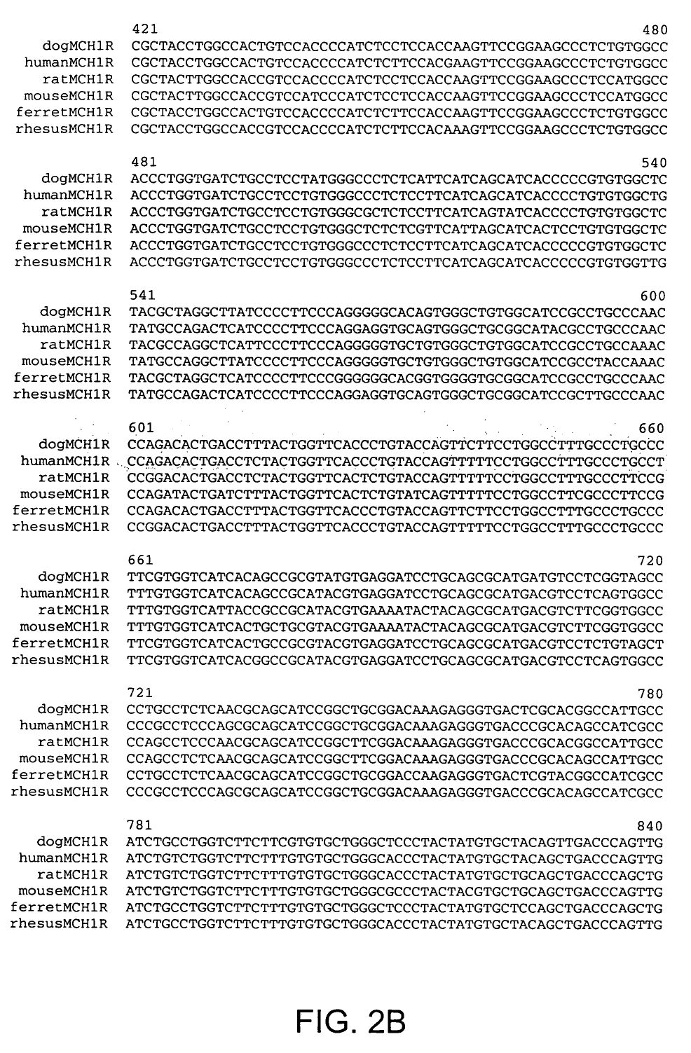

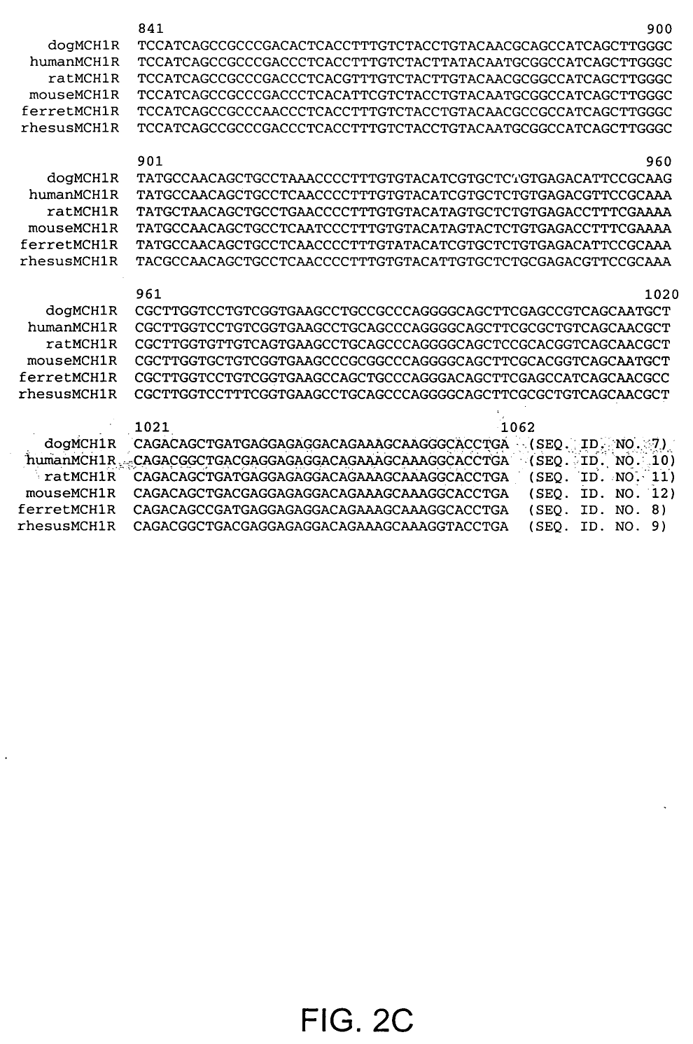

MCH-1R nucleic acid may contain a sequence region that is unique to either the dog, ferret, or rhesus monkey, or is present in two or more of these different animals. FIGS. 2A-2C illustrate a nucleotide sequence comparison of the dog, ferret, human, and rhesus monkey MCH-1R encoding nucleic acid. The sequence comparison provided in FIGS. 2A-2C identifies unique and common nucleotide sequences.

A “unique” nucleotide sequence differs from at least the human sequence, and preferably also from either the dog and ferret, dog and rhesus monkey, ferret and rhesus monkey, or dog and rhesus monkey. In embodiments of the present invention, the MCH-1R nucleic acid comprises or consists of a nucleotide sequence at least 18 bases in length that is unique for at least one of SEQ. ID. NOs. 7, 8, or 9 or the complement thereof. In additional embodiments the unique sequence is at least 36 or 54 bases in length.

A “common” nucleotide sequence is with respect to at least two of the dog, ferret, and rhesus monkey sequences. In an embodiment of the present invention, the MCH-1R nucleic acid has a nucleotide sequence at least 11 bases in length that is common to the dog, ferret, and rhesus monkey MCH-1R, but is not present in the human, rat, or mouse MCH-1R. Examples of such nucleic acid comprise a nucleotide sequence selected from group consisting of:

| | |
|---|---|
| SEQ. ID. NO. 14: | AACCTCACCTC; |
| SEQ. ID. NO. 15: | TTCATCAGCATCACCCCCGTG; |
| SEQ. ID. NO. 16: | CAACCCAGACACTGACCTTT; |
| SEQ. ID. NO. 17: | CTGCCCTTCGTGGTCATC; and |
| SEQ. ID. NO. 18: | CTCACCTTTGTCTACCT. |

Additional MCH-1R nucleic acid includes nucleic acid encoding a functional G-protein that responds to MCH where (1) the encoded G-protein has a sequence similarity of at least 98% with SEQ. ID. NOs. 1, 2, or 3, (2) the encoded G-protein has a sequence differing from SEQ. ID. NOs. 1, 2, 3 by up to 10 alterations; or (3) the nucleic acid has a sequence similarity of at least about 98% with SEQ. ID. NO. 7, 8, or 9. Sequence similarity for nucleic acid can be determined by FASTA. (Pearson 1990. Methods in Enzymology 183, 63-98, hereby incorporated by reference herein.) In one embodiment, sequence similarity is determined using the FASTA search program with the following parameters: MATRIX: BLOSUM50, GAP PENALTIES: open=−12; residue=−2.

The guidance provided in the present application can be used to obtain nucleic acid sequence encoding MCH-1R related receptors from different sources and to construct a receptor having MCH-1R activity. Obtaining nucleic acids encoding MCH-1R related receptors from different sources is facilitated using sets of degenerative probes and primers and by the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., Molecular Cloning, A Laboratory Manual, 2nd Edition, Cold Spring Harbor Laboratory Press, 1989. PCR cloning techniques are described, for example, in White, Methods in Molecular Cloning, volume 67, Humana Press, 1997.

MCH-1R probes and primers can be used to screen nucleic acid libraries containing, for example, genomic DNA or cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded by different combinations of nucleotide triplets or “codons”. Amino acids are encoded by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UWC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CWU

M=Met=Methionine: codon AUG

N=Asn=49Apar gine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUW

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., Molecular Cloning, A Laboratory Manual, 2 Edition, Cold Spring Harbor Laboratory Press, 1989.

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2 Edition, Cold Spring Harbor Laboratory Press, 1989, and Kacian, et al., U.S. Pat. No. 5,480,784.

MCH-1R Probes

Probes for MCH-1R contain a region that can specifically hybridize to MCH-1R target nucleic acid under appropriate hybridization conditions and can distinguish target nucleic acid from one or more non-target nucleic acids. Probes for MCH-1R can also contain nucleic acid that is not complementary to MCH-1R nucleic acid.

Preferably, non-complementary nucleic acid that is present has a particular purpose such as being a reporter sequence or being a capture sequence. However, additional nucleic acid need not have a particular purpose as long as the additional nucleic acid does not prevent the MCH-1R probe from distinguishing between target and non-target.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the Tm of the produced hybrid. The higher the Tm the stronger the interactions and the more stable the hybrid. Tm is affected by different factors well known in the art such as the degree of complementarity, the type of complementary bases present (e.g., A-T hybridization versus G-C hybridization), the presence of modified nucleic acid, and solution components. (E.g., Sambrook, et al., Molecular Cloning, A Laboratory Manual, 2 Edition, Cold Spring Harbor Laboratory Press, 1989.)

Stable hybrids are formed when the Tm of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6× SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Probes are composed of nucleic acids or derivatives thereof such as modified nucleic acid and peptide nucleic acid. Modified nucleic acid includes nucleic acid with one or more altered sugar groups, altered internucleotide linkages, and/or altered nucleotide purine or pyrirnidine bases. References describing modified nucleic acid include WO 98/02582, U.S. Pat. No. 5,859,221 and U.S. Pat. No. 5,852,188, each of which are hereby incorporated by reference herein.

Recombinant Expression

MCH-1R polypeptides can be expressed from recombinant nucleic acid in a suitable host or in a test tube using a translation system. Recombinantly expressed MCH-1R polypeptides are preferably used in assays to screen for compounds that bind to MCH-1R and modulate the activity of the receptor.

Preferably, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Expression vectors providing suitable levels of polypeptide expression in different hosts are well known in the art. Mammalian expression vectors well known in the art include pcDNA3 (Invitrogen), pMClneo (Stratagene), pXT1 (Stratagene), pSG5 (Stratagene), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), pCI-neo (Promega) and .lambda.ZD35 (ATCC 37565). Bacterial expression vectors well known in the art include pET11a (Novagen), lambda gt11 (Invitrogen), pcDNAII (Invitrogen), and pKK223-3 (Pharmacia). Fungal cell expression vectors well known in the art include pYES2 (Invitrogen), Pichia expression vector (Invitrogen). Insect cell expression vectors well known in the art include Blue Bac III (Invitrogen).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK.sup.-) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171).

To enhance expression in a particular host it may be useful to modify the sequence provided in SEQ. ID. NOs. 7, 8, or 9 to take into account codon usage of the host. Codon usage of different organisms are well known in the art. (See, Ausubel, Current Protocols in Molecular Biology, John Wiley, 1987-1998, Supplement 33 Appendix IC.)

Expression vectors may be introduced into host cells using standard techniques. Examples of such techniques include transformation, transfection, lipofection, protoplast fusion, and electroporation.

Nucleic acid encoding an MCH-1R polypeptide can be expressed in a cell without the use of an expression vector by, for example, creating or introducing a recombinant nucleic acid encoding a MCH-1R polypeptide into the cell genome. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection.

### MODULATING MCH-1R ACTIVITY

Using the present application as a guide compounds able to modulate MCH-1R can be obtained and used to achieve a beneficial effect in a patient. Beneficial effects can be obtained, for example, by altering weight or relieving stress using a compound active at MCH-1R.

Altering weight is particularly useful for gaining weight in an-under weight patient or losing weight in an over weigh patient. i additire,pl farm animals can be treated to gain weight. Under weight patients include those having a body weight about 10% or less, 20% or less, or 30% or less, than the lower end of a “normal” weight range or Body Mass Index (“BMI”). Over weight patients include those having a body weight about 10% or more, 20% or more, 30% or more, or 50% or more, than the upper end of a “normal” weight range or BMI. “Normal” weight ranges are well known in the art and take into account factors such as a patient age, height, and body type.

BMI measures your height/weight ratio. It is determined by calculating weight in kilograms divided by the square of height in meters. The BMI “normal” range is 19-22.

MCH-1R modulating compounds can be provided in a kit. Such a kit typically contains an active compound in dosage forms for administration. A dosage form contains a sufficient amount of active compound such that a beneficial effect can be obtained when administered to a patient during regular intervals, such as 1 to 6 times a day, during the course of 1 or more days. Preferably, a kit contains instructions indicating the use of the dosage form for weight reduction (e.g., to treat obesity or overweight) or stress reduction, and the amount of dosage form to be taken over a specified time period.

### DOSING FOR THERAPEUTIC APPLICATIONS

Guidelines for pharmaceutical administration in general are provided in, for example, Remington's Pharmaceutical Sciences 18^{th} Edition, Ed. Gennaro, Mack Publishing, 1990, and Modern Pharmaceutics 2^{nd} Edition, Eds. Banker and Rhodes, Marcel Dekker, Inc., 1990, both of which are hereby incorporated by reference herein.

MCH1R active compounds having appropriate functional groups can be prepared as acidic or base salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexdanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine.

MCH1R active compounds can be administered using different routes including oral, nasal, by injection, and transmucosally. Active ingredients to be administered orally as a suspension can be prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants.

When administered by nasal aerosol or inhalation, compositions can be prepared according to techniques well known in the art of pharmaceutical formulation. Such techniques can involve preparing solutions in saline, employing benzyl alcohol or other suitable preservatives, absorption promoters to enhance bioavailability, fluorocarbons, or other solubilizing or dispersing agents.

Routes of administration include intravenous (both bolus and infusion), intraperitoneal, subcutaneous, topical with or without occlusion, and intramuscular. Injectable solutions or suspensions known in the art include suitable non-toxic, parenterally-acceptable diluents or solvents, such as mannitol, 1,3-butanediol, water, Ringer's solution and isotonic sodium chloride solution. Dispersing or wetting and suspending agents, include sterile, bland, fixed oils, such as synthetic mono- or diglycerides; and fatty acids, such as oleic acid.

Rectal administration in the form of suppositories include the use of a suitable non-irritating excipient, such as cocoa butter, synthetic glyceride esters or polyethylene glycols. These excipients are solid at ordinary temperatures, but liquidify and/or dissolve in the rectal cavity to release the drug.

Suitable dosing regimens for the therapeutic applications of the present invention are selected taking into account factors well known in the art including age, weight, sex and medical condition of the patient; the severity of the condition to be treated; the route of administration; the renal and hepatic function of the patient; and the particular compound employed.

Optimal precision in achieving concentrations of drug within the range that yields efficacy without toxicity requires a regimen based on the kinetics of the drug's availability to target sites. This involves a consideration of the distribution, equilibrium, and elimination of a drug. The daily dose for a patient is expected to be between 0.01 and 1,000 mg per adult patient per day.

### EXAMPLES

Examples are provided below to further illustrate different features of the present invention. The examples also illustrate useful methodology for practicing the invention. These examples do not limit the claimed invention.

### Example 1

### Cloning Rhesus MCH-1R

The full-length coding sequence of rhesus MCH-1R was cloned as follows.

Two mix-base primers MCH3-1 (GGI ATG CCI TTY HTI ATH CAY CA; SEQ. ID. NO. 19) and MCH3-2 (ARY TGI ADI ACR TRR TAI GGI GC; SEQ. ID. NO. 20) were synthesized (H=A, C, or T, R=G or A, Y=T or C, I=inosine). Using a rhesus brain cDNA λgt10 library (Clontech, Palo Alto, Calif.) as a template, PCR was carried out with the primers and the products were cloned into pCR2.1-TOPO vector (Invitrogen, Carlsbad, Calif.). A clone was randomly picked up and sequenced with M13 forward and reverse primers by a dye-terminator method using ABI 377 sequencer (PE Biosystems, Foster City, Calif.). As a result, a partial sequence of rhesus MCH-1R cDNA was obtained.

Based on the above partial sequence, four primers, mMCH1R-1 (CACGGCCATGGATGCCAATAGTCAG; SEQ. ID. NO. 21), mMCH1R-2 (AAGAAGACCAGGCAGATGGCGATGG; SEQ. ID. NO. 22), mMCH1R-3 (TCACCAGCACCTACATCCTGACCGC; SEQ. ID. NO. 23), and mMCH1R4 (CAGGGCAAAGGCCAGGAAAAACTGG; SEQ. ID. NO. 24) were designed and synthesized. PCR-based RACE reactions were carried out using vector and gene specific primers. Employing the rhesus brain cDNA library, RACE reactions were firstly conducted with mMCH1R-1+gt10-1 (vector specific primer; AGTCAACACTTACGCCAAGAGCTGA; SEQ. ID. NO. 25) or gt10-3 (CGCCTCCATCAACAAACTMTCTrGTAT; SEQ. ID. NO. 26), and mMCH1R-2+gt10-1 or gt10-3. The PCR products from these reactions (primary reactions) were used as templates to carry out secondary PCR reactions using primers nested within the primary reactions, i.e., mMCH1R-3 (inside of mMCH1R-1)+gt10-2 (vector-specific, inside of gt10-1; TTAGTTTTACCGTTTTCGAGCTGCTCTA; SEQ. ID. NO. 27) or gt10-4 (vector-specific , inside of gt10-3; GATTGGGGGTAAATAACAGAGGTGG; SEQ. ID. NO. 28) for templates from mMCH1R-1-containing primary reactions, and mMCH1R-4 (inside of mMCH1R-2) +gt10-2 or gt10-4 for templates from mMCH1R-2-containing primary reactions. The amplified products were cloned and sequenced as mentioned above. All nucleotide sequence data were assembled using software, Sequencher (Gene Codes, Ann Arbor, MI), resulting in the identification of an open reading frame of 1062 nucleotides encoding a polypeptide of 353 amino acids and a stop codon. The cDNA containing the entire ORF sequence was cloned by PCR using the rhesus brain cDNA λgt10 library.

### Example 2

### Transient Expression of Rhesus MCH-1R

The entire coding sequence of rhesus MCH-1R was cloned into EcoRI-NotI site of pEF1lV5-HisB plasmid vector (Invitrogen, Carlsbad, Calif.). The resultant construct was transfected into HEK293T cells using LipofectAmine PLUS (Life Technologies, Rockville, Md.) according to the manufacture's instructions. Human embryonic kidney cells constitutively expressing SV40 large T antigen (HEK-293T) were maintained in Dulbecco's modified Eagle medium (Life Technologies) supplemented with 10% fetal bovine serum, 100 units/ml penicillin-G and 100 μg/ml streptomycin at 37° C. with 5% CO₂ in a humidified atmosphere.

The intracellular calcium ion concentration ([Ca²⁺]ᵢ) was measured fluorometrically using a Ca²⁺-sensitive fluorescent dye, fura-2. HEK293T cells transiently transfected with pEF1N5-HisB plasmid vector harboring rhesus MCH-1R cDNA were harvested by phosphate-buffered saline containing 2 mM EDTA 48 hours after transfection, and washed once with the assay buffer (Hanks' balanced salt solution containing 20 mM HEPES and 0.1% BSA, pH 7.4). The cells were suspended with the buffer containing 2 μM fura-2 acetoxymethylester (Dojin, for 60 minutes with gently shaking. The fura-2-loaded cells were washed twice with the buffer and re-suspended with the buffer to 1.0×10⁶ cells/ml. 0.5 ml of the resultant suspension was stirred continuously at 37° C. in a glass cuvette during the measurement. Two point five microliters of dimethyl sulfoxide (DMSO) solution of MCH (Peptide Institute, Osaka, Japan) or [Phe¹³,Tyr¹⁹]-MCH (Bachem, Bubendorf, Switzerland) was added into the cell suspension, and fluorescent intensity at an emission wavelength of 500 nm and excitation wavelengths of 340 and 380 nm was monitored with a CAF-110 intracellular ion analyzer (JASCO, Tokyo, Japan). Data were analyzed using the software GraphPad Prism Version 3.0 (GraphPad Software, Inc., San Diego, Calif., USA).

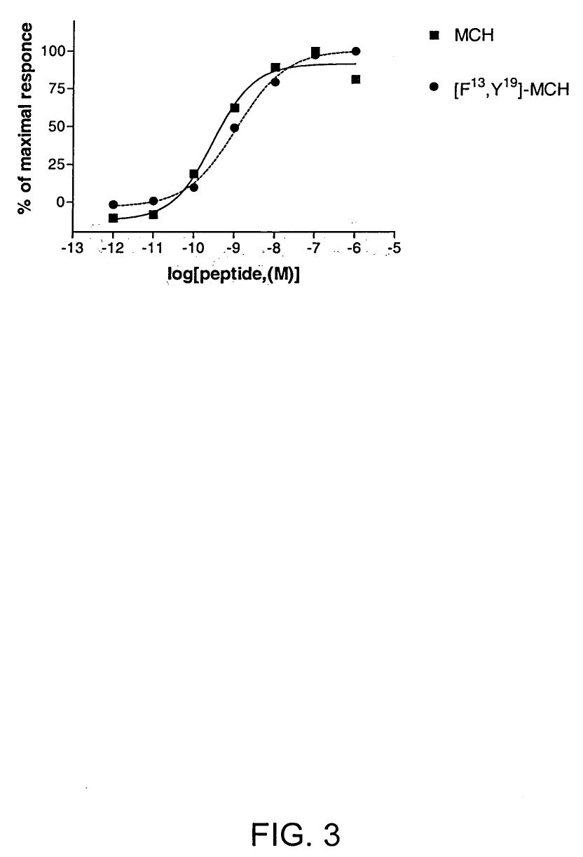

As shown in FIG. 3, both MCH and [Phe¹³,Tyr¹⁹]-MCH dose-dependently caused an increase in intracellular calcium levels in the HEK293T cells transiently expressing rhesus MCH-1R with potent efficacy (EC₅₀ of MCH and [Phe¹³,Tyr¹⁹]-MCH were calculated as 0.30 and 1.1 nM, respectively), but failed to induce detectable [Ca²⁺]ᵢ increase in the non-transfected cells (data not shown). These results confirm that the rhesus MCH-1R sequence encodes an active MCH receptor.

### Example 3

### MCH Binding Experiments

HEK293T cells were seeded into 24-well culture plates coated with poly-D-Lys at 1×10⁵ cells/well and were cultured during over-night. The adherent cells were transfected with pEF1/V5-HisB/rhesus MCH-1R plasmid (see Example 2). Forty-eight hours after transfection, the transfected monolayer cells were rinsed with the assay buffer (Hanks' balanced salt solution containing 20 mM HEPES, 0.2% BSA and 100 μg/ml bacitracin, pH 7.4). The cells were then incubated in 250 μl well of the same buffer with [¹²⁵I]-MCH (100 pM, NEN Life Science Products, Boston, Mass.) or [¹²⁵I]-[Phe¹³,Tyr¹⁹]-MCH (100 pM, NEN Life Science Products) for 30 minutes at 37° C. After the incubation, the cells were washed three times with the ice cold assay buffer and lysed with 500 μl/well of 2 M NaOH. The lysates were transferred into test tubes and the cell- bound radioactivity was measured by a COBLA Quantum γ-counter (Packard Instrument, Meriden, CT). Nonspecific binding was defined in the presence of 1 μM cold MCH or [Phe¹³,Tyr¹⁹]-MCH for the corresponding radio ligands.

[¹²⁵I-MCH and [¹²⁵I]-[Phe¹³,Tyr¹⁹]-MCH bound to the HEK293T cells expressing rhesus MCH-1R with good windows. Specific binding was not observed into mock transfected cells (Table 1.)

TABLE 1

| | [¹²⁵I]-MCH binding | | [¹²⁵I]-[Phe¹³,Tyr¹⁹]-MCH | |
|---|---|---|---|---|
| | (cpm) | | binding (cpm) | |
| | | Nonspecific | | Nonspecific |
| | Total binding | binding | Total binding | binding |
| Rhesus | 2542.8 | 275.1 | 5353.0 | 1524.1 |
| MCH-1R | 2893.4 | 721.6 | 5542.3 | 2023.5 |
| | 2315.5 | 662.8 | 6345.9 | 1029.6 |
| Mock | 209.9 | 172.0 | 708.9 | 539.7 |
| | 215.8 | 180.9 | 663.3 | 537.7 |
| | 239.8 | 184.1 | 771.5 | 557.8 |

HEK293T cells transfected with pEF1IV5-HisB/MCH1R plasmid were harvested by phosphate-buffered saline containing 2 mM EDTA 48 hours after transfection, and washed once with the assay buffer (Hanks' balanced salt solution containing 20 mM HEPES, 0.2% BSA and 100 μg/ml bacitracin, pH 7.4). The cells (4×10⁵ cells/tube) were then incubated in 250 μl/tube of the same buffer with 100 pM [121I]- MCH for 30 minutes at 37° C. After the incubation, bound and free [²⁵I]-MCH were separated by filtration using a Unifilter GF/B glass filter (Packard Instrument) presoaked with 0.3% polyethylenimine. The remaining radioactivity on the filter was quantitated using a TopCount HTS (Packard instrument) with a Microscint 0 scintillation cocktail (Packard instrument). Specific binding was defined as the difference between total binding and nonspecific binding in the presence of 1 μM cold MCH. Data were analyzed using GraphPad Prism Version 3.0.

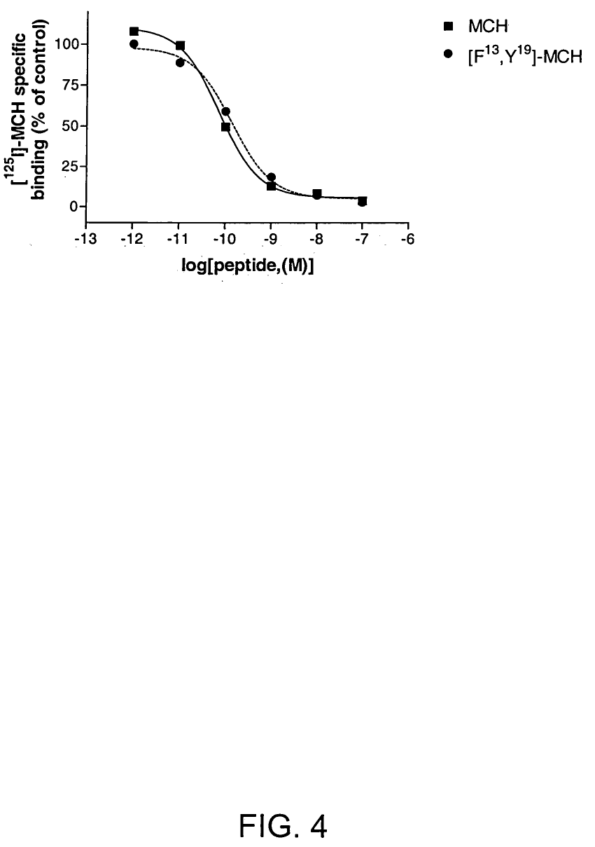

As shown in FIG. 4, cold MCH and [Phe¹³,Tyr¹⁹]-MCH dose-dependently inhibited the [¹²⁵I]-MCH binding to the rhesus MCH-1R expressed on HEK293T cells. IC₅₀ values of MCH and [Phe¹³,Tyr¹⁹]-MCH in this experiment were calculated as 0.074 and 0.14 nM, respectively.

### Example 4

### Dog MCH-1R Cloning

The dog MCH-1R was cloned by first obtaining a 700 bp fragment of dog MCH-1R encoding part of the receptor and then using the fragment to obtain a longer length sequence. The dog MCH-1R fragment was generated by PCR from a dog hypothalamus library glycerol stock using primers perfectly conserved between rat, mouse and human MCH-1R. The forward primer was RMH-MCH-1F 5′-TTCATGATCCACCAGCTCATGGG-3′ (SEQ. ID. NO. 29). The reverse primer was RMH-MCH-tgaR 5′-TCAGGTGCCTTTGCTTTCTGTCC-3 (SEQ. ID. NO. 30). The dog MCH-1R fragment was isolated using gel electrophoresis. The resulting band TA cloned and sequenced confirmed.

The dog MCH-1R fragment was used as a probe on colony hybridization screen of a dog hypothalamus cDNA library. Numerous unspliced cDNA clones were identified consisting only Exon2 of dog MCH-1R going into the intron at the splice junction. One clone was identified possessed Exon 1 of dog MCH-1R and thus the remaining 80 bp of sequence was determined.

### Example 5

### Ferret MCH-1R Cloning

Ferret MCH-1R was cloned from a ferret brain cDNA library. A 26 weeks old castrated male ferret was sacrificed, dissected and the brain tissue was immediately frozen in liquid nitrogen. Brain tissue was homogenized under liquid nitrogen using a ceramic mortar and pestle. Total brain RNA was isolated as described by Chomczynski et al., Anal. Biochem. 162:156-159, 1987.

For subsequent isolation of polyadenylated messenger RNA, a PolyA Tract MRNA Isolation system (Promega, Madison, Wis.) was used according to the manufacturers protocol. First strand cDNA synthesis was primed using a mix of oligo dT and random hexamer primers as well as SuperScript II reverse transcriptase (Invitrogen Life Technologies, Carlsbad, Calif.) according to the manufacturers instructions. After second strand synthesis and adapter ligation (BstXIl/EcoRI) the cDNA was cloned into the plasmid vector pcDNA3.1 Hygro(+) (Invitrogen Life Technologies, Carlsbad, Calif.).

Full length cDNA for the ferret MCH-1R was isolated by screening 1.5×10⁶ colonies at high stringency (0.1× SSC, 65° C.) with a partial ferret MCH-1R probe. The MCH-1R probe was previously isolated by RT-PCR. A total of 18 clones were identified and the sequence of a clone with a cDNA insert of 2.6 kb was determined.

For subsequent analysis of the binding and functional properties of the ferret MCH-1R the coding region was amplified by PCR using primers: F1R/Eco/Kozak/ATG and F1R/TGA/Xba/rev). PCR conditions were as follows: 95° C. 1 minute, (95° C. 30 seconds, 65° C. 1 minute, 68° C. 2 minutes,) 30 cycles, 68° C. 3 minutes. The coding region was transferred into the plasmid vector pcDNA3.1hygro+ and pCIneo (Promega, Madison, Wis.). Primer sequences:

| | | |
|---|---|---|
| F1R/Eco/Kozak/ATG: | CGGAATTCGCCGCCATGGACCTGGGAGCCTCGCTGC | (SEQ. ID. NO. 31) |
| F1R/TGA/Xba/rev: | GCTCTAGATCAGGTGCCTTTGCTTTCTGTCCTC | (SEQ. ID. NO. 32) |

Other embodiments are within the following claims. While several embodiments have been shown and described, various modifications may be made without departing from the spirit and scope of the present invention.

1. A purified polypeptide comprising the amino acid sequence of SEQ ID NO: 13.

2. The polypeptide of claim 1, wherein said polypeptide comprises a sequence selected from the group consisting of SEQ ID NO: 1, SEQ ID NO: 2, and SEQ ID NO: 3.

3. The polypeptide of claim 2, wherein said polypeptide consists of SEQ ID NO: 1.

4. The polypeptide of claim 2, wherein said polypeptide consists of SEQ ID NO: 2.

5. The polypeptide of claim 2, wherein said polypeptide consists of SEQ ID NO: 3.

6. A purified nucleic acid comprising a nucleotide sequence encoding the polypeptide of claim 1.

7. A purified nucleic acid comprising a nucleotide sequence selected from the group consisting of: SEQ ID NO: 14, SEQ ID NO: 15, SEQ ID NO: 16, SEQ ID NO: 17, SEQ ID NO: 18, the complement of SEQ ID NO: 14, the complement of SEQ ID NO: 15, the complement of SEQ ID NO: 16, the complement of SEQ ID NO: 17, and the complement of SEQ ID NO: 18.

8. The purified nucleic acid of claim 7, wherein said nucleotide sequence comprises SEQ ID NO: 14 or the complement thereof.

9. The purified nucleic acid of claim 7, wherein said nucleotide sequence comprises SEQ ID NO: 15 or the complement thereof.

10. The purified nucleic acid of claim 7, wherein said nucleotide sequence comprises SEQ ID NO: 16 or the complement thereof.

11. The purified nucleic acid of claim 7, wherein said nucleotide sequence comprises SEQ ID NO: 17 or the complement thereof.

12. The purified nucleic acid of claim 7, wherein said nucleotide sequence comprises SEQ ID NO: 18 or the complement thereof.

13. A recombinant nucleic acid comprising a nucleotide sequence encoding the amino acid sequence claim 1.

14. The recombinant nucleic acid of claim 13, wherein said recombinant nucleic acid is an expression vector.

15. A recombinant cell comprising an expression vector encoding the amino acid sequence of claim 1 functionally coupled to a promoter recognized by said cell.

16. A recombinant cell made by a process comprising the step of introducing into said cell the expression vector of claim 14.

17. A method of measuring the ability of a compound to affect MCH-1R activity comprising the steps of:
a) contacting a recombinant cell with said compound, wherein said recombinant cell comprises a recombinant nucleic acid expressing a functional MCH-1R that comprises the amino acid sequence of claim 1; and
b) measuring MCH-1R activity.

18. The method of claim 17, wherein said method further comprises the use of an MCH-1R agonist.

19. A method of preparing a MCH-1R polypeptide comprising the step of growing the recombinant cell of claim 15 under conditions wherein said polypeptide is expressed from said expression vector.

20. A recombinant nucleic acid comprising the nucleotide sequence of claim 7.
